(19) United States
(12) Patent Application Publication
(10) Pub. No.: US 2026/0345657 A1
(43) Pub. Date: Nov. 5, 2026

(54) STABLE PHARMACEUTICAL FORMULATIONS OF GEMCITABINE

(71) Applicant: Ingenus Pharmaceuticals, LLC, Orlando, FL (US)

(72) Inventors: Mukteeshwar GANDE, Monroe, NJ (US); Praveen Reddy BILLA, Flanders, NJ (US); Samir Chimanlal MEHTA, Morrisville, NC (US); Venkata S. VARANASI, Fogelsville, PA (US)

(73) Assignee: Ingenus Pharmaceuticals, LLC, Orlando, FL (US)

(21) Appl. No.: 19/187,628

(22) Filed: Apr. 23, 2025

(51) Int. Cl. A61K 31/7068; A61K 9/00; A61K 9/08; A61K 31/337; A61K 33/243; A61P 35/00

(52) U.S. Cl. CPC A61K 31/7068; A61K 9/0019; A61K 9/08; A61K 31/337; A61K 33/243; A61P 35/00

(57) ABSTRACT

Described herein are stable, preservative-free, multi-dose gemcitabine injection formulations that maintain sterility and stability over multiple withdrawals. The formulations comprise gemcitabine, or a pharmaceutically acceptable salt thereof, an alkalizing agent, and water for injection, and have a pH between 2.0 and 3.0. Methods of making and using the formulations are also described.

### FIELD OF THE DISCLOSURE

The present disclosure relates to stable ready-to-dilute multi-dose injectable formulations comprising gemcitabine, or a pharmaceutically acceptable salt thereof; an alkalizing agent; and a pharmaceutically acceptable fluid. The disclosure further encompasses processes for preparing these formulations and methods for treating a patient suffering from cancer.

### BACKGROUND

Gemcitabine is a well-known chemotherapeutic agent used in the treatment of various cancers. It is the active ingredient in the commercially available product, GEMZAR® for Injection by Lilly USA, currently manufactured and marketed by JIANGSU HANSOH PHARMACEUTICAL GROUP CO LTD. GEMZAR® is a sterile, white to off-white lyophilized powder available in single-dose vials of 200 mg and 1 g, intended for intravenous use only. Each vial of GEMZAR® contains lyophilized gemcitabine hydrochloride (equivalent to gemcitabine), mannitol, and sodium acetate. Hydrochloric acid and/or sodium hydroxide may be added for pH adjustment. GEMZAR® requires reconstitution prior to intravenous infusion. To obtain the required dose, which is calculated based on the patient's body surface area (BSA), healthcare professionals often need to reconstitute multiple vials to deliver a single therapeutic dose. According to the prescribing information, the reconstitution process involves mixing the powder with a specified quantity of 0.9% Sodium Chloride Injection USP to obtain a clear, free of particulate matter, colorless to light straw-colored solution with a concentration of 38 mg/mL. The reconstituted solution is then further diluted with 0.9% Sodium Chloride Injection USP to a minimum final concentration of at least 0.1 mg/mL before administration. The reconstitution process is time-consuming, tedious, and requires complex steps to be performed under aseptic conditions with extreme caution. Animal studies have shown that dermal absorption of gemcitabine solution can be fatal, as noted in the GEMZAR® package insert. This underscores the risk healthcare professionals face if proper precautions are not taken during the reconstitution process.

One of the primary challenges with the commercially available form(s) of gemcitabine is the complexity of its administration. The reconstitution procedure is cumbersome, requiring several steps that add significant time to dose preparation, while also posing cytotoxic safety risks to healthcare professionals handling the drug. Further, GEMZAR® is designed for single use only. According to the prescribing information, any unused portion of the drug must be discarded. As a result, this single-dose lyophilized formulation presents several limitations, including cumbersome and time-consuming aseptic reconstitution, potential for dosing errors, exposure of healthcare professionals to cytotoxic vapors, and wastage of remaining drug after the dose has been administered. Additionally, the lyophilization process itself is time-consuming and incurs significant expenses.

Moreover, the reconstituted product and reconstituted-diluted product exhibit limited stability. They must be stored under refrigerated conditions for no longer than 24 hours and discarded thereafter. When the aqueous solution of gemcitabine powder is stored at room temperature for an extended period of time, unknown impurities may form. The relatively rapid formation of degradation products is widely considered one of the factors preventing the availability of long-term stable aqueous gemcitabine formulations. According to ASHP, gemcitabine hydrochloride diluted in 0.9% sodium chloride and stored at 22 °C did not prevent the antimicrobial growth of four microorganisms (Enterococcus faecium, Staphylococcus aureus, Pseudomonas aeruginosa, and Candida albicans) inoculated into the solution. See, ASHP INJECTABLE DRUG INFORMATION, Gemcitabine Hydrochloride at pp. 761-62. C. albicans remained viable for 120 hours, while the others remained viable for 24 hours. As a result, the solution fails the microbial challenge test and shows that gemcitabine hydrochloride in saline solution does not act as an antimicrobial agent under the specified conditions.

Another commercially available gemcitabine formulation is provided by Hospira as a sterile solution in vials containing 200 mg, 1 g, or 2 g of gemcitabine (equivalent to 227.7 mg, 1.139 g, or 2.227 g of gemcitabine HCl), with each mL containing 38 mg of gemcitabine free base. Hydrochloric acid and/or sodium hydroxide may have been added for pH adjustment. This formulation is supplied in single-dose vials for intravenous use, and the required dose is withdrawn based on the calculated patient-specific dose. Prior to administration, the appropriate volume is diluted with 0.9% Sodium Chloride Injection to achieve a minimum final concentration of at least 0.1 mg/mL. The diluted gemcitabine solution must be used within 24 hours if stored at controlled room temperature (20° to 25 °C), and any unused portion must be discarded leading to wastage of drug.

Another commercial formulation by Accord is a sterile, clear, colorless to pale yellow solution containing 100 mg of gemcitabine free base (equivalent to 113.85 mg of gemcitabine hydrochloride), 250 mg PEG-300, 150 mg propylene glycol, and 16 mg sodium hydroxide in dehydrated alcohol. Sodium hydroxide and/or hydrochloric acid may be added for pH adjustment. This product is supplied as a multi-dose vial, allowing the remaining solution to be reused after the initial withdrawal. It can be stored for up to 28 days before being discarded. However, the Accord product includes additional excipients like polyethylene glycol, propylene glycol, and sodium hydroxide in dehydrated alcohol, which may lead to side effects. Moreover, since the product is concentrated (100 mg/mL), any variation in withdrawal volume during dilution or administration could result in under- or over-dosage, which is particularly critical since the dose is calibrated based on body surface area.

The recommended dosage of gemcitabine injection is primarily determined by patient-specific factors, particularly body surface area (BSA), as well as the therapeutic indication. The dosage of gemcitabine is individualized to achieve optimal therapeutic outcomes while minimizing adverse effects. Since gemcitabine doses are tailored to each patient based on BSA and the specific treatment protocol, wastage is common when commercially available vial sizes do not align with the calculated patient-specific doses. For example, the Hospira gemcitabine product is provided in single-dose vials, which presents a challenge when the calculated dose does not match the quantity in the vial. This often results in leftover drug that must be discarded, leading to inefficiencies and wastage. In addition, gemcitabine is frequently administered in combination with other chemotherapy agents, especially in the treatment of advanced pancreatic or ovarian cancer, which can exacerbate the wastage problem when vial sizes do not align with patient-specific dosing.

Therefore, there is a clear need for a stable, multi-dose gemcitabine injection formulation with minimal excipients to avoid these issues.

Without being bound by theory, it is known that when formulating injectable dosage forms, it is essential to ensure that the solubility of the active ingredient is maintained throughout the shelf life, preventing precipitation during storage. Stability is another critical factor that must be carefully considered in the formulation of injectable drugs. There must be a balance between the stability and solubility of the active ingredient to ensure the drug remains effective and safe throughout its shelf life.

In the case of gemcitabine injection, the stability and solubility of gemcitabine salt in injectable formulations are highly pH dependent. Maintaining an optimal pH is crucial for preserving the chemical integrity of gemcitabine and minimizing degradation. At acidic pH, the molecule becomes more susceptible to hydrolytic degradation, leading to the formation of impurities such as 2′-Deoxy-2′,2′-difluorouridine. At higher pH levels, gemcitabine's stability is compromised, leading to the formation of undesirable degradation products like gemcitabine α-anomer and cytosine. Moreover, the solubility of gemcitabine salt in aqueous solutions is pH-dependent, with better solubility observed at pH 4-5 (the pH of tumor interstitial fluid) compared to pH 7.4 (the pH of blood). As such, it is essential to formulate gemcitabine at an optimal pH to maintain its stability and solubility over long periods of storage.

Jansen et al. studied the degradation kinetics of gemcitabine hydrochloride in aqueous solutions at pH 3.2 and identified several new degradation products. See, Jansen et al., The Degradation of the Antitumor Agent Gemcitabine Hydrochloride in an Acidic Aqueous Solution at pH 3.2 and Identification of Degradation Products. J. Pharm. Sci., 2000, 89 (7), 885-891. Xu et al. evaluated the physical and chemical stability of gemcitabine hydrochloride solutions under various storage conditions, considering different concentrations, packaging, and temperature. See, Xu et al., Physical and Chemical Stability of Gemcitabine Hydrochloride Solutions. J. Am. Pharm. Assoc. (Wash). 1999 July-August; 39(4): 509-13. US 2006/0089329 and US 2012/0129799 address ready-to-use gemcitabine formulations, with the former describing a concentrated gemcitabine solution and the latter detailing non-aqueous gemcitabine preparations. However, all prior attempts either offer short-term storage products, single-dose formulations, or ready-to-use solutions.

Therefore, there is a critical need for the development of stable, ready-to-dilute (RTD), multi-dose gemcitabine formulations that not only maintain their stability for an extended period of time but also maintain their chemical integrity, sterility, and efficacy throughout their shelf life. Such formulations would significantly reduce the potential for degradation and the formation of impurities, which are common issues with current products. By ensuring that these formulations can withstand long-term storage without compromising the quality or therapeutic effect, healthcare professionals would benefit from a more efficient and safer treatment option, minimizing wastage and the need for frequent reconstitution. Ultimately, this would provide a more convenient, cost-effective, and practical solution for patients and healthcare providers alike.

To address the challenges outlined above, the present inventors have developed a multi-dose vial formulation containing gemcitabine salt. This formulation is designed to allow multiple withdrawals from a single vial without compromising the stability, sterility, or efficacy of the drug. The present invention specifically addresses the need for a pharmaceutically stable, long-term storage gemcitabine formulation that preserves the solution dosage form and prevents the degradation of active ingredients. Moreover, this multi-dose formulation helps mitigate drug wastage, especially in clinical settings where multiple doses may be needed for different patients or during the course of treatment. This ultimately results in a more efficient and cost-effective approach to administering the drug, benefiting both healthcare providers and patients.

As gemcitabine formulations have limited stability, and considering the degradation products that form during storage, the development of a long-term stable, multi-dose gemcitabine dosage form, such as a ready-to-dilute injection with minimal excipients, is highly challenging. The present inventors conducted various experiments to develop a gemcitabine formulation that addresses these, and other, issues.

### SUMMARY

Some embodiments of the present invention provide a multi-dose pharmaceutical formulation comprising gemcitabine, or a pharmaceutically acceptable salt thereof; an alkalizing agent; and a pharmaceutically acceptable fluid.

Other embodiments of the present invention provide a multi-dose pharmaceutical formulation comprising gemcitabine, or a pharmaceutically acceptable salt thereof; an alkalizing agent; and a pharmaceutically acceptable fluid, wherein the formula exhibits stability when stored at 2 °C to 8 °C for an extended period of time.

Further embodiments of the present invention provide a multi-dose pharmaceutical formulation comprising gemcitabine, or a pharmaceutically acceptable salt thereof; an alkalizing agent; and a pharmaceutically acceptable fluid; wherein the formulation maintains stability during long-term storage at 2 °C to 8 °C. In some embodiments, the formulations of the present invention are substantially free, or free, of preservatives and are provided in a multi-dose vial that maintains stability during long-term storage at 2 °C to 8 °C.

As used herein, the term “long-term storage” refers to a period of time in excess of twenty-four months, optionally up to thirty-six months, e.g., the period of time that lyophilized gemcitabine formulations remain in storage prior to reconstitution.

In some embodiments, the formulations of the present invention comprise gemcitabine, or a pharmaceutically acceptable salt thereof; and a pharmaceutically acceptable fluid, wherein the pharmaceutically acceptable fluid comprises less than about five excipients, optionally less than four excipients, or less than three excipients. Some embodiments of the present invention provide injectable formulations comprising gemcitabine, or a pharmaceutically acceptable salt thereof; and a pharmaceutically acceptable fluid, wherein the pharmaceutically acceptable fluid comprises two excipients.

Yet other embodiments of the present invention provide a pharmaceutical formulation comprising gemcitabine, or a pharmaceutically acceptable salt thereof; having a precisely controlled pH, ensuring that the gemcitabine salt remains in a stable and soluble form throughout its shelf life, thus maintaining its therapeutic efficacy and minimizing the formation of degradation products.

In some embodiments, the present invention provides a pharmaceutical formulation comprising gemcitabine, or a pharmaceutically acceptable salt thereof; an alkalizing agent; and a pharmaceutically acceptable fluid, which reduces drug wastage compared to other commercially available gemcitabine products.

Still further embodiments of the present invention provide a pharmaceutical formulation comprising gemcitabine, or a pharmaceutically acceptable salt thereof, that eliminates the cumbersome process of reconstituting dry powder, is easy to administer, minimizes exposure of healthcare providers to the drug's cytotoxic vapours during reconstitution, and reduces the risk of dosing errors during administration.

In some embodiments, the present invention provides a primary packaging system that ensures the stability and sterility of the multi-dose gemcitabine formulation, allowing for multiple withdrawals from a single vial without compromising the drug's efficacy or sterility. The primary packaging also ensures that microbial sterility is maintained even after multiple insertions following the first withdrawal of the formulation.

In some embodiments, the present invention provides a stable multi-dose pharmaceutical injectable liquid formulation of gemcitabine, or a pharmaceutically acceptable salt thereof, optionally at a concentration of about 35 mg/mL to about 40 mg/mL (optionally, about 38 mg/mL), formulated with an alkalizing agent and a pharmaceutically acceptable fluid. In some embodiments, the formulation is maintained at a pH between 2.0 and 3.0 to ensure the stability and solubility of the active ingredient. In certain embodiments, the formulation is free of preservatives and stabilizers and is stored in a multi-dose vial with an oxygen-impermeable and light-resistant container-closure system, which ensures the stability and sterility of the product over multiple withdrawals.

In some embodiments, the formulations of the present invention remain stable for at least about 24 months.

As used herein, the term “stable” refers to an injectable gemcitabine formulation containing less than 1.5% wt. % of 2′-Deoxy-2′,2′-difluorouridine and less than 1.0 wt. % of other impurities after 24 months, when stored at 2 °C to 8 °C.

In some embodiments, the formulations of the present invention contain less than 0.1 wt. % of gemcitabine degradation products, such as cytosine and/or gemcitabine α-anomer after 24 months when stored at 2 °C to 8 °C. In certain embodiments, the formulations of the present invention are stable for at least about 14 days after first use. In some embodiments, the formulations of the present invention are stable for up to about 10 withdrawals from a multi-use vial. In some embodiments, the formulations of the present invention are stable for 14 days after one withdrawal from a multi-use vial. In some embodiments, the formulations of the present invention are stable for 14 days after two withdrawals from a multi-use vial. In some embodiments, the formulations of the present invention are stable for 14 days after three withdrawals from a multi-use vial. In some embodiments, the formulations of the present invention are stable for 14 days after four withdrawals from a multi-use vial. In some embodiments, the formulations of the present invention are stable for 14 days after five withdrawals from a multi-use vial. In some embodiments, the formulations of the present invention are stable for 14 days after six withdrawals from a multi-use vial. In some embodiments, the formulations of the present invention are stable for 14 days after seven withdrawals from a multi-use vial. In some embodiments, the formulations of the present invention are stable for 14 days after eight withdrawals from a multi-use vial. In some embodiments, the formulations of the present invention are stable for 14 days after nine withdrawals from a multi-use vial. In some embodiments, the formulations of the present invention are stable for 14 days after ten withdrawals from a multi-use vial. In some embodiments, the formulations of the present invention are stable for 14 days after eleven withdrawals from a multi-use vial. In some embodiments, the formulations of the present invention are stable for 14 days after twelve withdrawals from a multi-use vial. In some embodiments, the formulations of the present invention are stable for 14 days after thirteen withdrawals from a multi-use vial. In some embodiments, the formulations of the present invention are stable for 14 days after fourteen withdrawals from a multi-use vial. In some embodiments, the formulations of the present invention are stable for 14 days after greater than about ten withdrawals from a multi-use vial.

Some embodiments of the present invention provide a formulation in various vial pack sizes, including 200 mg/5.26 mL, 1 g/26.3 mL, and 2 g/52.6 mL, along with a ready-to-dilute option using 0.9% NaCl, where gemcitabine is present at a concentration of about 0.1 mg/mL in the diluted form. In some embodiments, the formulations of the present invention significantly reduce drug wastage compared to commercially available gemcitabine products, thereby improving economic efficiency and reducing the burden of unused drug disposal in clinical settings.

Still further embodiments of the present invention provide a method for preparing the pharmaceutical formulation, which involves compounding, filtration, and filling into vials under sterile conditions with nitrogen purging during manufacturing. In some embodiments, the multi-dose formulations of the present invention are suitable for use in treating various cancers, including pancreatic cancer, ovarian cancer, breast cancer, and non-small cell lung cancer, when administered in combination with other chemotherapy agents.

### DETAILED DESCRIPTION

Gemcitabine is a nucleoside metabolic inhibitor. The chemical name of gemcitabine HCl is 2′-deoxy 2′,2′-difluorocytidine monohydrochloride (β-isomer). The structural formula is as follows:

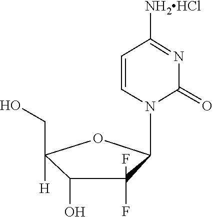

Gemcitabine hydrochloride has the molecular formula of C₉H₁₁F₂N₃O₄·HCl and a molecular weight of 299.66 g/mol. Gemcitabine hydrochloride is an off-white powder. It is soluble in water, slightly soluble in methanol, and practically insoluble in ethanol and polar organic solvents.

Gemcitabine is a nucleoside metabolic inhibitor that kills cells undergoing DNA synthesis and blocks the progression of cells through the G1/S-phase boundary.

The term “gemcitabine” is used in a broad sense to include not only “gemcitabine” per se, but also its pharmaceutically acceptable derivatives thereof. Suitable pharmaceutically acceptable derivatives include pharmaceutically acceptable salts, pharmaceutically acceptable solvates, pharmaceutically acceptable hydrates, pharmaceutically acceptable anhydrates, pharmaceutically acceptable enantiomers, pharmaceutically acceptable esters, pharmaceutically acceptable isomers, pharmaceutically acceptable polymorphs, pharmaceutically acceptable prodrugs, pharmaceutically acceptable tautomers, pharmaceutically acceptable complexes etc.

In some embodiments, the present invention provides a stable multi-dose pharmaceutical formulation comprising gemcitabine, or a pharmaceutically acceptable salt, isomer, racemate, enantiomer, hydrate, solvate, metabolite, polymorph, or mixture thereof. Preferably, gemcitabine is the hydrochloride salt thereof.

In some embodiments, the present invention relates to the development of stable multi-dose pharmaceutical formulations comprising gemcitabine or its pharmaceutically acceptable derivatives, wherein the concentration of the gemcitabine in the formulation is 38 mg/mL.

In some embodiments, the formulations of the present invention further comprise an alkalizing agent to maintain the pH of the solution within the optimal range of 2.0 to 3.0, ensuring both the stability and solubility of gemcitabine. Maintaining this pH range is critical to preventing the hydrolytic degradation of gemcitabine, which can lead to the formation of undesirable byproducts such as 2′-Deoxy-2′,2′-difluorouridine. Notably, the formulation does not utilize a combination of alkalizing and acidifying agents, as such a combination may result in the formation of salts that can unpredictably alter the pH of the solution, potentially compromising the stability and therapeutic efficacy of the drug. Instead, sodium hydroxide (NaOH) is preferably used as the alkalizing agent, allowing precise pH control without introducing the risks associated with acidic conditions. The use of NaOH ensures that gemcitabine remains stable throughout its shelf life while preventing unwanted degradation and maintaining the formulation's suitability for multi-dose administration. By optimizing pH adjustment through NaOH solely, embodiments of the present invention provide a multi-dose gemcitabine injection with enhanced stability and efficacy, offering a reliable treatment option for patients.

In one embodiment, the pharmaceutical formulation described herein is free of acidifying agents, bulking agents, buffering agents and combinations thereof.

As per another embodiment, the formulation of present invention is preservative free.

The multi-dose gemcitabine formulations of present invention have the ability to withstand the microbial challenge test, even in the absence of preservatives. In some embodiments, the formulation of the present invention, comprising gemcitabine, or a pharmaceutically acceptable salt thereof; an alkalizing agent; and a pharmaceutically acceptable fluid, has ability to maintain sterility and inhibit microbial growth over its intended shelf life when stored under suitable conditions.

The microbial challenge test involves the introduction of common test microorganisms, such as Candida albicans, Aspergillus niger, Escherichia coli, Pseudomonas aeruginosa, and Staphylococcus aureus, to the formulation. In certain embodiments, even without the addition of preservatives, the formulations of the present invention can effectively resist microbial contamination and retain their sterility over time, ensuring that it remains safe for use in clinical settings or after multiple withdrawals from a single vial. The microbial challenge tests indicate that the formulations of the present invention effectively inhibit the growth of bacteria, yeast, mold, and maintain microbial sterility across multiple withdrawals without requiring preservatives or stabilizers. According to this embodiment, the formulation is capable of sustaining the microbial challenge test and remains safe for patient use during its intended shelf life. This ability to remain free from microbial growth, even without preservatives, ensures that the gemcitabine formulation is suitable for long-term storage and multiple-dose administration, providing an enhanced safety profile compared to other formulations that require preservatives for microbial control. The formulations of the present application further comprise a pharmaceutically acceptable fluid.

As used herein, the terms “pharmaceutically acceptable fluid” or “pharmaceutically acceptable vehicle” are used for dissolution of gemcitabine hydrochloride and keeping the drug is solution form throughout the intended storage duration. These terms can be used interchangeably. Suitable pharmaceutically acceptable fluids include, but are not limited to, aqueous solutions, water, saline, D₅W, or mixtures thereof. In several embodiments of the invention, the pharmaceutical formulations include water for injection as a pharmaceutically acceptable fluid.

The term “composition” or “formulation” or “dosage form” has been employed interchangeably for purposes of the present invention and refers to a pharmaceutical composition which is suitable for administration to a patient or subject. The subject can be an animal, preferably a mammal, more preferably a human.

The term “administration” or “administering,” as used herein, refers to a method of giving a dosage of a compound or composition to a subject, such as an animal, preferably a mammal, more preferably a human via a suitable mode of administration. In various embodiments as will be disclosed herein, the preferred mode of administration can be parenteral administration, injectable formulations administered via any route including intramuscular, intravenous, or subcutaneous.

The term “ready-to-dilute” used herein refers to aqueous preconcentrates, solutions which require a single step of dilution with an aqueous diluent fluid such as water for injection or saline before administration. The term “ready-to-dilute” is distinguished from lyophilized products that require two steps, a first step of reconstitution to form a preconcentrate and then a second step where the preconcentrate is subjected to dilution with an aqueous infusion fluid.

In some embodiments, the ready-to-dilute formulation may be provided as a single vial containing a gemcitabine formulation as described herein. In other embodiments, the ready-to-dilute formulation may be accompanied by a pharmaceutically acceptable diluent in a separate container (i.e., a dual vial formulation).

In some embodiments, the ready-to-dilute formulation is a parenteral formulation. In some embodiments, the ready-to-dilute parenteral formulation of the present invention avoids the inconvenience of reconstituting a concentrated parenteral formulation into infusion diluents prior to infusion; and eliminates the risk of potential calculation or dilution errors and the risk of microbiological contamination during handling.

In some embodiments, the pH of the formulations of the present invention is maintained precisely by the use of alkalizing agent in the range from about 2.0 to about 3.0. In certain embodiments, sodium hydroxide is used to maintain the pH of the formulation in the range from about 2.0 to about 3.0.

In accordance with some embodiments of the present invention, the process for preparing the stable, preservative-free, multi-dose gemcitabine injection formulation comprises carefully controlled steps to ensure sterility, stability, and suitability for multiple withdrawals. In other embodiments, the process includes the following key stages such as compounding wherein the required quantity of gemcitabine hydrochloride is weighed and dissolved in water for injection (WFI) under controlled stirring conditions to achieve complete dissolution. In certain embodiments, the pH of the solution is adjusted to a range of 2.0 to 3.0 using sodium hydroxide (NaOH) as an alkalizing agent. In further embodiments, the volume of the solution is adjusted to the desired level with WFI to obtain the target concentration of about 38 mg/mL of gemcitabine. Yet other embodiments provide methods wherein the solution is continuously purged with nitrogen gas to minimize oxygen exposure, reducing oxidative degradation and ensuring long-term stability. In some embodiments, the next step involves filtration wherein the compounded solution undergoes a two-stage filtration process. In some embodiments, the first stage comprises a bioburden reduction filtration through a pre-sterilized 0.45 μm filter or 0.22 micron filter to remove particulates and bioburden; and the second stage comprises a sterilizing filtration through a sterile-grade 0.22 μm filter to ensure microbial sterility. In certain embodiments, the filtration is carried out under aseptic conditions in a classified cleanroom environment. In other embodiments, the last step involves filling, stoppering, and sealing of the solution. In further embodiments, the sterile-filtered solution is filled into pre-sterilized, light-resistant, glass vials in precise fill volumes. In some embodiments, each vial is sealed with a stopper and a crimped aluminium flip-off cap to ensure product integrity.

In certain embodiments, the present invention provides a specialized primary packaging component, which plays a crucial role in maintaining the sterility, stability, and suitability of a preservative-free, multi-dose gemcitabine injection formulation. In other embodiments, the formulation is stored in glass vials, which provide a light-resistant and chemically inert environment, ensuring the stability of the active pharmaceutical ingredient. In further embodiments, to prevent oxygen ingress and microbial contamination during multiple withdrawals, the vial is sealed with a low-permeability elastomeric stopper. This stopper is specifically selected for its oxygen-impermeable properties, which help maintain the integrity of the formulation without requiring additional preservatives. This stopper not only prevents microbial contamination upon repeated needle punctures but also minimizes oxygen exposure, thereby preserving the chemical integrity of gemcitabine throughout its shelf life. As a result, the formulation remains suitable for use over multiple dosing sessions, providing greater flexibility in clinical practice. Healthcare providers can withdraw and administer the precise amount required for each patient, avoiding overfill wastage while maintaining a consistent therapeutic profile.

In some embodiments, the present invention provides a method for administering a stable multi-dose pharmaceutical injectable liquid formulation in accordance with the present invention. In certain embodiments, the method comprises withdrawing a required volume corresponding to a prescribed dose from the multi-dose vial under aseptic conditions and administering it to a patient. In other embodiments, following the initial withdrawal, the remaining formulation is stored within the original packaging/carton at refrigerated conditions, specifically between 2 °C and 8 °C. In some embodiments, the remaining contents of the vial are stable and can be used for up to at least 14 days after the first withdrawal. During this period, the formulation may be utilized for subsequent dosing, either for the same patient or for additional patients, provided that each withdrawal is conducted aseptically and the vial is handled according to recommended pharmaceutical guidelines for multi-dose usage. Further, medical care workers are suitably advised about the multi dose utility of the present invention in the form of package label insert, label instructions on vial and carton which forms another embodiment of present invention. According to the present invention, the medical care worker or healthcare workers can be physician, clinical staff, nurse who is trained in preparation and administration of intravenous drugs.

Additionally, the vial is further secured with a crimped aluminium flip-off cap, ensuring product integrity and minimizing the risk of contamination. This optimized container-closure system extends the shelf life of the formulation and enables safe and effective multi-dose administration.

As discussed herein, the present invention provides a stable, preservative-free, multi-dose gemcitabine injection formulation, which allows for multiple withdrawals from a single vial without compromising sterility or efficacy. Unlike traditional single-use vials that result in drug wastage, the multi-dose formulations of the present invention ensure efficient drug utilization, reducing unnecessary disposal and overall treatment costs. In some embodiments, the formulations of the present invention are provided in multi-dose vials and stored under controlled refrigeration (2 °C to 8 °C) for at least about twenty-four months and are stable and sterile after multiple withdrawals.

By enabling multiple doses from a single vial, the present invention significantly enhances dose accuracy, economic efficiency, and hospital or clinical workflow management, ultimately benefiting both patients and healthcare systems.

Certain embodiments of the present invention significantly reduce drug wastage by enabling the reuse of unused formulation from a multi-dose vial, unlike conventional single-use formulations that must be discarded after a single withdrawal. In some embodiments, the multi-dose formulations of the present invention allow multiple withdrawals from a single vial while maintaining sterility, stability, and efficacy for at least twenty-four months. The advanced oxygen-impermeable elastomeric stopper ensures that sterility is preserved across multiple doses, eliminating the need for unnecessary disposal of unused drug. In some embodiments, the formulations of present invention provide 100% reduction in drug wastage compared to commercially available single-dose pack sizes optimizing drug utilization and significantly reducing the economic and environmental burden of pharmaceutical waste.

As used herein, “no wastage” refers to either zero or practically insignificant quantity of left over drug post maximum extraction from the multidose vial.

As used herein, the term “extended period of time” refers to the duration for which the multi-dose vials containing an injectable formulation comprising gemcitabine, or a pharmaceutically salt thereof, retains its chemical stability, sterility, and efficacy under specified storage conditions (e.g., at least twenty-four months). Under refrigerated storage conditions (2 °C to 8 °C), the formulations of the present invention maintain stability for at least 24 months, with impurity levels consistently within acceptable limits as determined by HPLC analysis. Furthermore, the formulation retains sterility and stability for at least 14 days after first puncture, enabling at the most 10 withdrawals while preventing microbial contamination and degradation.

Some embodiments of the present invention provide a multi-dose gemcitabine injection formulation in three different pack sizes to accommodate varying dosage requirements and treatment regimens. In some embodiments, the formulation is available in vial configurations such that 200 mg/5.26 mL provided in glass vial of capacity 10 mL, 1 g/26.3 mL provided in glass vial of capacity 30 mL, and 2 g/52.6 mL provided in glass vial of capacity 100 mL wherein the gemcitabine formulation has a concentration of 38 mg/mL. The varying pack sizes ensure that healthcare providers can select an appropriate vial size based on patient-specific dosing needs, which are typically determined using body surface area (BSA) calculations. These pack sizes are designed to minimize drug wastage by enabling multiple withdrawals from a single vial while maintaining sterility for at least 14 days after first puncture. The availability of different vial sizes enhances dose accuracy, cost-effectiveness, and inventory management, reducing the burden of unused drug disposal in hospital and clinical settings.

The multi-dose stable pharmaceutical formulation of the present invention comprising gemcitabine hydrochloride, an alkalizing agent and a pharmaceutically acceptable fluid can be further diluted to concentrations as low as 0.1 mg/mL with 0.9% Sodium Chloride Injection, USP. In certain embodiments, the formulation of the present invention maintains the stability after dilution with 0.9% Sodium Chloride Injection, USP for up to 24 hours at controlled room temperature of about 20 °C to about 25 °C.

Some embodiments of the present invention provide a method for treating patients suffering from cancer by administering a formulation of gemcitabine as described herein in therapeutic dose as calculated based on the body surface area. In certain embodiments, the present invention provides methods wherein the gemcitabine injection is administered as a single agent in treatment of pancreatic cancer. In other embodiments, a formulation of present invention is administered in combination with carboplatin for the treatment of advanced ovarian cancer that has relapsed at least 6 months after completion of platinum-based therapy. In yet other embodiments, a formulation of the present invention is administered in combination with paclitaxel, for first-line treatment of metastatic breast cancer after failure of prior anthracycline-containing adjuvant chemotherapy, unless anthracyclines were clinically contraindicated. Still further embodiments of the present invention provide methods wherein a formulation is administered in combination with cisplatin for the treatment of non-small cell lung cancer.

In some embodiments, the present invention provides methods of using a pharmaceutical formulation comprising gemcitabine hydrochloride as described herein for treating cancer comprising: advising a medical care worker using a packaging label insert along with a printed label on the vial and carton about the multi dose utilization; wherein the multi-dose vial containing the pharmaceutical formulation can be used multiple times for the same or different patient(s) within about 14 days after first use when stored at from about 2 °C to about 8 °C. In certain embodiments, the packaging and labelling comply with regulatory guidelines for multi-dose injectable products.

The following examples are for the purpose of illustration of the invention only and are not intended to limit the scope of the present invention in any manner whatsoever.

### EXAMPLES

### Example 1

Table 1 (below) describes an exemplary formulation of gemcitabine hydrochloride RTD injection.

TABLE 1

| S. No. | Ingredients | Quantity (mg/mL) |
|---|---|---|
| 1 | Gemcitabine hydrochloride | 43.2 |
| 2 | Sodium hydroxide | q.s. to adjust pH to 2.6 |
| 3 | Water for injection | q.s to 1 mL |

The formulation described in Table 1 (above) can be prepared by the following steps:

Compounding: gemcitabine hydrochloride was weighed and added to water for injection. pH was adjusted with sodium hydroxide to 2.6 such that the API is completely dissolved. Finally, the solution is made up to volume of batch size.

Filtration: the solution was filtered with sterile grade A 0.22 μm/0.45 μm filter to reduce the bioburden followed by second aseptic filtration step using grade A 0.22 μm filter.

Filling: The sterile filtered solution was filled into vials at the target fill volume range, stoppered, and sealed with flip-off seals. The vials were washed externally to remove any traces, if present.

Purging: nitrogen is used as an inert gas during the formulation's manufacturing, filtration, and filling steps.

As used herein, the term “purging” refers to the process by which a gas (e.g., nitrogen gas) is introduced into water for injection, compounding mass or solution in a closed system (e.g., a vial). For example, bubbling or otherwise passing the desired gas through a solution under atmospheric pressure to saturate the liquid and headspace with optimum quantities of the gas (e.g., nitrogen).

### Example 2

The stability of Gemcitabine formulation of present invention was evaluated as a function of pH in the range of 2.0 to 3.0. The Gemcitabine Injection formulation of present invention at a concentration of 38 mg of gemcitabine/mL were manufactured and adjusted to approximate pH values of 2.0±0.2, 2.5±0.2 and 3.0±0.2 filled in 10 mL moulded vials with a fill volume of 10 mL, overlaid with nitrogen and stoppered with stoppers and sealed. These vials were exposed to 80 °C for 1, 3, 4, and 6 hours. These samples from the three pH conditions were comparatively evaluated for description, pH, assay, and related substances against each other and, in comparison, with initial samples. The results of the study are provided in the table below.

Stability Test results at pH 2.0 at 80 °C.

TABLE 2.1

| | | Results | | | | |
|---|---|---|---|---|---|---|
| P | Test Parameters | Initial | 1 hr | 3 hr | 4 hr | 6 hr |
| 1 | Description | Clear | Clear | Clear | Clear | Clear |
| | | colourless | colourless | colourless | colourless | colourless |
| | | solution | solution | solution | solution | solution |
| 2 | pH | 2.05 | 1.96 | 1.96 | 1.98 | 1.98 |
| 3 | Assay | 100.1% | 99.4% | 96.9% | 96.5% | 95.0% |
| 4 | Related substances | | | | | |
| | Cytosine | 0.000% | 0.001% | 0.001% | 0.001% | 0.001% |
| | Gemcitabine α | 0.002% | 0.002% | 0.002% | 0.001% | 0.001% |
| | anomer | | | | | |
| | 2¹-Deoxy-2¹ 2¹ | 0.078% | 0.693% | 2.249% | 2.875% | 3.940% |
| | Difluoro Uridine | | | | | |
| | Single Max. | 0.003% | 0.004% | 0.004% | 0.003% | 0.003% |
| | Impurity | | | | | |
| | Total Impurities | 0.01% | 0.01% | 0.01% | 0.01% | 0.01% |
| | (Excluding 2¹- | | | | | |
| | Deoxy-2¹ 2¹ | | | | | |
| | Difluoro Uridine | | | | | |

Stability Test results at pH 2.5 at 80 °C.

TABLE 2.2

| | | Results | | | | |
|---|---|---|---|---|---|---|
| S. No. | Test Parameters | Initial | 1 hr | 3 hr | 4 hr | 6 hr |
| 1 | Description | Clear | Clear | Clear | Clear | Clear |
| | | colourless | colourless | colourless | colourless | colourless |
| | | solution | solution | solution | solution | solution |
| 2 | pH | 2.52 | 2.46 | 2.40 | 2.50 | 2.52 |
| 3 | Assay | 100.3% | 100.1% | 97.3% | 96.1% | 96.2% |
| 4 | Related substances | | | | | |
| | Cytosine | 0.000% | 0.000% | 0.000% | 0.001% | 0.001% |
| | Gemcitabine α | 0.002% | 0.001% | 0.002% | 0.001% | 0.001% |
| | anomer | | | | | |
| | 2¹-Deoxy-2¹ 2¹ | 0.075% | 0.636% | 1.971% | 2.649% | 3.563% |
| | Difluoro Uridine | | | | | |
| | Single Max. | 0.004% | 0.003% | 0.003% | 0.004% | 0.003% |
| | Impurity | | | | | |
| | Total Impurities | 0.01% | 0.01% | 0.01% | 0.01% | 0.01% |
| | (Excluding 2¹- | | | | | |
| | Deoxy-2¹ 2¹ | | | | | |
| | Difluoro Uridine | | | | | |

Stability Test results at pH 3.0 at 80 °C.

TABLE 2.3

| | | Results | | | | |
|---|---|---|---|---|---|---|
| S. No. | Test Parameters | Initial | 1 hr | 3 hr | 4 hr | 6 hr |
| 1 | Description | Clear | Clear | Clear | Clear | Clear |
| | | colourless | colourless | colourless | colourless | colourless |
| | | solution | solution | solution | solution | solution |
| 2 | pH | 2.95 | 2.93 | 2.97 | 2.96 | 2.99 |
| 3 | Assay | 99.4% | 98.8% | 97.2% | 96.3% | 95.1% |
| 4 | Related substances | | | | | |
| | Cytosine | 0.000% | 0.001% | 0.001% | 0.001% | 0.001% |
| | Gemcitabine α anomer | 0.001% | 0.001% | 0.001% | 0.001% | 0.001% |
| | 2¹-Deoxy-2¹ 2¹ Difluoro | 0.073% | 0.539% | 1.612% | 2.233% | 3.118% |
| | Uridine | | | | | |
| | Single Max. Impurity | 0.004% | 0.003% | 0.003% | 0.003% | 0.003% |
| | Total Impurities | 0.01% | 0.01% | 0.01% | 0.01% | 0.01% |
| | (Excluding 2¹-Deoxy- | | | | | |
| | 2¹ 2¹ Difluoro Uridine | | | | | |

The results described in Tables 2.1, 2.2 and 2.3 (above) illustrate there was no significant difference across 3 different pH levels and are in the comparative range. Hence, the pH range of 2.0 to 3.0 is determined to be acceptable.

### Example 3: Microbial Challenge Test

This test involves introducing specific microorganisms to the product to check if the growth of the microorganisms is inhibited ensuring that the formulation remains safe for use over its shelf life.

Common test organisms include Candida albicans (yeast), Aspergillus niger (mold), Escherichia coli (gram-negative bacterium), Pseudomonas aeruginosa (a gram-negative bacterium), and Staphylococcus aureus (gram-positive bacterium). The product is inoculated with a known concentration of each selected microorganism. The inoculated product is then incubated under conditions that are favourable for microbial growth. At specific time intervals (e.g., 7, 14 and 28 days), samples are taken from the product and analyzed to determine the number of surviving microorganisms. The reduction in the number of viable microorganisms is the evaluation criteria. A significant reduction in microbial count indicates effective preservation. The acceptance criteria for injection formulation is as follows:
Bacteria: Not less than a 1.0 log reduction from the initial count at 7 days, and no increase from the 7-day count at 14 and 28 days.
Yeasts and Molds: No increase from the initial count at 7, 14, and 28 days

A gemcitabine injection formulation in accordance with certain embodiments of the present invention was subjected to microbial challenge test as per USP and incubation parameters are Bacteria: 72 hours to 5 days at 30 °C to 35 °C, Yeast: Five to seven days at 20 °C to 25 °C, Mold: Five to seven days at 20 °C to 25 °C.

Acceptance Criteria for inoculum: Between 1×105 and 1×106 cfu per ml of the product.

Acceptance Criteria for bacterial results: Not less than a 1.0 log reduction from the initial calculated count at 7 days, not less than a 3.0 log reduction from the initial count at 14 days, and no increase from the 14 days' count at 28 days.

Acceptance Criteria for yeast and molds results: No increase from the initial calculated count at 7, 14, and 28 days. Note: No increase is defined as not more than 0.5 log 10 unit higher than the previous value.

The results of the formulation of present invention are as detailed below: The Results of Microbial challenge test of formulation of present invention are as detailed below in Table 3.

TABLE 3

| | | S. aureus | E. coli | P. aeruginosa | C. albicans | A. niger |
|---|---|---|---|---|---|---|
| Initial | Control Count | 1.3E+05 | 1.2E+05 | 2.2E+05 | 3.3E+05 | 5.5E+05 |
| | (CFU/ml) | | | | | |
| | Test Sample | 1.5E+02 | <100 | <100 | 3.0E+04 | 9.5E+05 |
| | Count (CFU/ml) | | | | | |
| Day 0 | Log reduction | 2.9 | >3.1 | >3.3 | 1.0 | −0.2 |
| | Test Sample | <100 | <100 | <100 | 4.0E+04 | 5.8E+05 |
| | Count (CFU/ml) | | | | | |
| Day 7 | Log reduction | >3.1 | >3.1 | >3.3 | 0.9 | 0.0 |
| | Test Sample | <100 | <100 | <100 | 1.8E+04 | 6.0E+05 |
| | Count (CFU/ml) | | | | | |
| Day 14 | Log reduction | >3.1 | >3.1 | >3.3 | 1.3 | 0.0 |
| | Test Sample | <100 | <100 | <100 | 1.9E+04 | 7.5E+05 |
| | Count (CFU/ml) | | | | | |
| Day 28 | Log reduction | >3.1 | >3.1 | >3.3 | 1.3 | −0.1 |
| | Final Status | Pass | Pass | Pass | Pass | Pass |

Staphylococcus aureus = S. aureus
Escherichia Coli = E. coli
Pseudomonas Aeruginosa = P. aeruginosa
Candida Albicans = C. albicans
Aspergillus Niger = A. niger

The results described in Table 3 (above), demonstrate that an exemplary gemcitabine hydrochloride injection of the present invention passed the microbial challenge test and thus remains free of any microbial growth which makes the formulation suitable for long term storage without use of any preservative.

### Example 4: Multi-Vial Use Study

An In Use Stability study was performed to establish multiple dose capability of the formulation of present invention. This study was conducted on two batches (Batch A having pack size: 2 g/52.6 mL and Batch B having pack size 20 0 mg/5.26 mL) against a control over a period of 14 days, where in the batches under study were punctured 10 times using 21-gauge needle. There was no puncture for the control sample. Samples were drawn at T 0, T 7 and T 14 days' time interval. Evaluation was performed on Physical, Chemical & Microbiological parameters. The physical parameters include: description; particulate matter; color absorbance; and light transmission. The chemical parameters include: pH; related substances; and assay. The microbiological parameters include: sterility; and bacterial endotoxins.

The results of the same are described in Table 4 (below).

TABLE 4

| | | | Unpunctured vials | | | | | |
|---|---|---|---|---|---|---|---|---|
| | Test | | (control sample) | | | Punctured vials | | |
| S. No. | parameters | Specification | T₀ days | T₇ days | T₁₄ days | T₀ days | T₇ days | T₁₄ days |
| 1 | Description | Clear, | Clear, | Clear, | Clear, | Clear, | Clear, | Clear, |
| | | colorless to | colorless | colorless | colorless | colorless | colorless | colorless |
| | | light straw- | solution | solution | solution | solution | solution | solution |
| | | colored | | | | | | |
| | | solution | | | | | | |
| 2 | pH | 2.0-3.0 | 2.7 | 2.7 | 2.7 | 2.7 | 2.7 | 2.8 |
| 3 | Assay of | NLT 95.0%- | 100.8% | 100.6% | 100.2% | 100.4% | 100.9% | 100.5% |
| | Gemcitabine | NMT 105.0% | | | | | | |
| | (by HPLC) | | | | | | | |
| 4 | Related substances by HPLC | | | | | | | |
| | Cytosine | NMT0.10% | Below | Below | Below | Below | Below | Below |
| | | | LOD | LOD | LOD | LOD | LOD | LOD |
| | Gemcitabine | NMT0.10% | Below | Below | Below | Below | Below | Below |
| | α anomer | | LOQ | LOQ | LOQ | LOQ | LOQ | LOQ |
| | 2¹-Deoxy-2¹ | NMT1.50% | 0.7% | 0.7% | 0.7% | 0.7% | 0.7% | 0.7% |
| | 2¹ Difluoro | | | | | | | |
| | Uridine | | | | | | | |
| | Single Max. | NMT 0.10% | Below | Below | Below | Below | Below | Below |
| | Impurity | | LOQ | LOQ | LOQ | LOQ | LOQ | LOQ |
| | Total | NMT 0.50% | Below | Below | Below | Below | Below | Below |
| | Impurities | | LOQ | LOQ | LOQ | LOQ | LOQ | LOQ |
| | (Excluding | | | | | | | |
| | 2¹-Deoxy- | | | | | | | |
| | 2¹ 2¹ | | | | | | | |
| | Difluoro | | | | | | | |
| | Uridine | | | | | | | |
| 5 | Particulate matter | | | | | | | |
| | Visible | Solution is | Solution is | Solution is | Solution is | Solution is | Solution is | Solution is |
| | particles | free from | free from | free from | free from | free from | free from | free from |
| | | particles of | particles of | particles of | particles of | particles of | particles of | particles of |
| | | foreign | foreign | foreign | foreign | foreign | foreign | foreign |
| | | matter | matter | matter | matter | matter | matter | matter |
| | Sub-visible particles: Number of particles per container, diameter of which is | | | | | | | |
| | ≥10 μm | NMT 6000 | 6 | 4 | 7 | 7 | 16 | 12 |
| | ≥25 μm | NMT600 | 1 | 0 | 0 | 0 | 0 | 0 |
| 6. | Sterility | Sterile | Sterile | Sterile | Sterile | Sterile | Sterile | Sterile |
| 7. | Bacterial | NMT 0.05 | <0.03 | <0.03 | <0.03 | <0.03 | <0.03 | <0.03 |
| | endotoxins | USP EU/ mg | EU/mg | EU/mg | EU/mg | EU/mg | EU/mg | EU/mg |
| | | gemcitabine | | | | | | |
| 8. | Color | NMT 0.1 | 0.0 | 0.0 | 0.0 | 0.0 | 0.0 | 0.0 |
| | absorbance | | | | | | | |
| | (at 430 nm) | | | | | | | |
| 9. | Light | NLT 96.0% | 100.4% | 100.8% | 100.1% | 100.4% | 100.5% | 100.4% |
| | transmission | | | | | | | |
| | (at 650 nm) | | | | | | | |

NLT = not less than
NMT = not more than
LOD = limit of detection
LOQ = limit of quantitation
Limit of Quantitation (LOQ) Values:
Cytosine: 0.0032%;
Gemcitabine alpha anomer: 0.0030%;
2¹-Deoxy-2¹ 2¹ Difluoro Uridine: 0.0018%

As demonstrated by the results described in Table 4 (above), both batches of two different pack sizes are stable for all physical, chemical and microbiological parameters after 10 multiple insertions using Gauge 21 Needle across the time intervals of T0, T7 and T14 days which endorses the multidose utility of this invention.

### Example 5: Reduction in Wastage of Drug

The following calculations are performed for different doses, which are determined based on the body surface area (BSA) and the recommended dose as per the relevant indications. The optimum combination of commercially available products is selected to deliver the required therapeutic dose. The number of vials of various pack sizes from the present invention are also considered, ensuring an efficient combination. A comparison is made between the wastage of drugs when using commercial products and the wastage associated with the pack sizes of the present invention. Since the pack sizes in the present invention are multi-dose vials, any remaining drug after withdrawal for the therapeutic dose can be reused, resulting in zero wastage.

### Example 5.1: Treatment of Ovarian Cancer—Co-Administration with Carboplatin

Dose: 1000 mg/m²

TABLE 5.1

| | | Commercial product pack sizes | | | Product of present invention | |
|---|---|---|---|---|---|---|
| Patient BSA | Recommended | | Total drug | Drug waste | pack sizes | |
| (m²) | Dose (mg) | Vials needed | (mg) | (mg) | Vials needed | Drug waste |
| 1.5 | 1500 | 1vial of 1000 mg/26.3 mL + | 1600 | 100.0 | 1 vial of 2000 mg/52.6 mL | No waste |
| | | 3 vials of 200 mg/ 5.26 mL | | | | |
| 1.6 | 1600 | 1vial of 1000 mg/26.3 mL + | 1600 | 0.0 | 1 vial of 2000 mg/52.6 mL | |
| | | 3 vials of 200 mg/ 5.26 mL | | | | |
| 1.7 | 1700 | 1vial of 1000 mg/26.3 mL + | 1800 | 100.0 | 1 vial of 2000 mg/52.6 mL | |
| | | 4 vials of 200 mg/ 5.26 mL | | | pack size | |
| 1.8 | 1800 | 1vial of 1000 mg/26.3 mL + | 1800 | 0.0 | 1 vial of 2000 mg/52.6 mL | |
| | | 4 vials of 200 mg/ 5.26 mL | | | | |

### Example 5.2: Treatment of Breast Cancer (w/Paclitaxel)

Dose: 1250 mg/m²

TABLE 5.2

| | | Commercial product pack sizes | | | Product of present invention | |
|---|---|---|---|---|---|---|
| Patient BSA | Recommended | | Total drug | Drug waste | pack sizes | |
| (m²) | Dose (mg) | Vials needed | (mg) | (mg) | Vials needed | Drug waste |
| 1.5 | 1875 | 1 vial of 2000 mg/52.6 mL | 2000.0 | 125.0 | 1 vial of 2000 mg/52.6 mL | No waste |
| 1.6 | 2000 | 1 vial of 2000 mg/52.6 mL | 2000.0 | 0.0 | 1 vial of 2000 mg/52.6 mL | |
| 1.7 | 2125 | 1 vial of 2000 mg/52.6 mL + | 2200.0 | 75.0 | 1 vial of 2000 mg/52.6 mL + | |
| | | 1 vial of 200 mg/5.26 mL | | | 1 vial of 200 mg/5.26 mL | |
| 1.8 | 2250 | 1 vial of 2000 mg/52.6 mL + | 2400.0 | 150.0 | 1 vial of 2000 mg/52.6 mL + | |
| | | 2 vials of 200 mg/5.26 mL | | | 1 vial of 1000 mg | |
| | | | | | mg/ 26.3 mL | |

### Example 5.3: Treatment of NSCLC (w/Cisplatin) 28 Days Regimen

Dose: 1000 mg/m²

TABLE 5.3

| | | Commercial product pack sizes | | | Product of present invention | |
|---|---|---|---|---|---|---|
| Patient | Recommended | | Total drug | Drug waste | pack sizes | |
| BSA (m²) | Dose (mg) | Vials needed | (mg) | (mg) | Vials needed | Drug waste |
| 1.5 | 1500 | 1vial of 1000 mg/26.3 mL + | 1600.0 | 100.0 | 1 vial of 2000 mg/52.6 mL | No waste |
| | | 3 vials of 200 mg/5.26 mL | | | | |
| 1.6 | 1600 | 1vial of 1000 mg/26.3 mL + | 1600.0 | 0.0 | 1 vial of 2000 mg/52.6 mL | |
| | | 3 vials of 200 mg/5.26 mL | | | | |
| 1.7 | 1700 | 1vial of 1000 mg/26.3 mL + | 1800.0 | 100.0 | 1 vial of 2000 mg/52.6 mL | |
| | | 4 vials of 200 mg/5.26 mL | | | | |
| 1.8 | 1800 | 1vial of 1000 mg/26.3 mL + | 1800.0 | 0.0 | 1 vial of 2000 mg/52.6 mL | |
| | | 4 vials of 200 mg/5.26 mL | | | | |

### Example 5.4: Treatment of NSCLC (w/Cisplatin) 28 Days Regimen

Dose: 1250 mg/m²

TABLE 5.4

| | | Commercial product pack sizes | | | Product of present invention | |
|---|---|---|---|---|---|---|
| Patient | Recommended | | Total drug | Drug waste | pack sizes | |
| BSA (m²) | Dose (mg) | Vials needed | (mg) | (mg) | Vials needed | Drug waste |
| 1.5 | 1875 | 1 vial of 2000 mg/52.6 mL | 2000.0 | 125.0 | 1 vial of 2000 mg/52.6 mL | No waste |
| 1.6 | 2000 | 1 vial of 2000 mg/52.6 mL | 2000.0 | 0.0 | 1 vial of 2000 mg/52.6 mL | |
| 1.7 | 2125 | 1 vial of 2000 mg/52.6 mL + | 2200.0 | 75.0 | 1 vial of 2000 mg/52.6 mL | |
| | | 1 vial of 200 mg/5.26 mL | | | | |
| 1.8 | 2250 | 1 vial of 2000 mg/52.6 mL + | 2400.0 | 150.0 | 1 vial of 2000 mg/52.6 mL + | |
| | | 2 vials of 200 mg/5.26 mL | | | 1 vial of 1000 mg/26.3 mL | |

### Example 5.5: Pancreatic Cancer (Monotherapy)

Dose: 1000 mg/m²

TABLE 5.5

| | | Commercial product pack sizes | | | Product of present invention | |
|---|---|---|---|---|---|---|
| Patient BSA | Recommended | | Total drug | Drug waste | pack sizes | |
| (m2) | Dose (mg) | Vials needed | (mg) | (mg) | Vials needed | Drug waste |
| 1.5 | 1500 | 1vial of 1000 mg/26.3 mL + | 1600.0 | 100.0 | 1 vial of 2000 mg/52.6 mL | No waste |
| | | 3 vials of 200 mg/5.26 mL | | | | |
| 1.6 | 1600 | 1vial of 1000 mg/26.3 mL + | 1600.0 | 0.0 | 1 vial of 2000 mg/52.6 mL | |
| | | 3 vials of 200 mg/5.26 mL | | | | |
| 1.7 | 1700 | 1vial of 1000 mg/26.3 mL + | 1800.0 | 100.0 | 1 vial of 2000 mg/52.6 mL | |
| | | 4 vials of 200 mg/5.26 mL | | | | |
| 1.8 | 1800 | 1vial of 1000 mg/26.3 mL + | 1800.0 | 0.0 | 1 vial of 2000 mg/52.6 mL | |
| | | 4 vials of 200 mg/5.26 mL | | | | |

The data described in the foregoing Tables (5.1 to 5.5) clearly demonstrates a significant reduction in drug wastage achieved through the use of the pack sizes of the present invention. By utilizing multi-dose vials of the present invention, any remaining drug after withdrawal for the therapeutic dose can be effectively utilized resulting in zero wastage across all indicated therapeutic regimens.

In contrast, this is not possible with commercially available single-dose vials. In various indications and doses, the wastage of product with commercially available single-dose pack sizes ranges from 75 mg to 150 mg. However, with the multi-dose pack sizes of the present invention, the corresponding wastage is zero. As a result, certain embodiments of the present invention, even without preservatives, provide a 100% reduction in drug wastage compared to commercially available single-dose pack sizes.

1. A stable aqueous multi-dose pharmaceutical injectable liquid formulation, comprising:
a) gemcitabine or a pharmaceutically acceptable salt thereof in a concentration of 38 mg/mL;
b) an alkalizing agent; and
c) water for injection;
wherein the pH of the formulation is maintained between 2.0 and 3.0;
wherein the formulation is substantially free of preservatives and stabilizers, and is stored in a multi-dose vial with an oxygen-impermeable and light-resistant container-closure system;
wherein the formulation remains stable for at least 24 months when stored at from about 2 °C to about 8 °C;
wherein the concentration of 2′-Deoxy-2′,2′-difluorouridine, a degradation product of gemcitabine, does not exceed 1.5 wt. %;
wherein the total impurity level, excluding 2′-Deoxy-2′,2′ difluorouridine, does not exceed 1.0 wt. % as determined by HPLC analysis;
wherein the formulation maintains its sterility and stability for 14 days after first use when stored at from about 2 °C to about 8 °C; and
wherein the multi-dose vial is accessed up to 10 times during the 14 days.

2. The pharmaceutical formulation according to claim 1, wherein the pharmaceutically acceptable salt of gemcitabine is a hydrochloride salt.

3. The pharmaceutical formulation according to claim 1, wherein the alkalizing agent comprises sodium hydroxide.

4. (canceled)

5. The pharmaceutical formulation according to claim 1, wherein the total impurity level, excluding 2′-Deoxy-2′,2′-difluorouridine, does not exceed 0.3%.

6. The pharmaceutical formulation according to claim 1, wherein the formulation contains less than 0.1 wt. % of the gemcitabine degradation products, cytosine and gemcitabine α-anomer.

7. A pharmaceutical product comprising:
a pharmaceutical formulation according to claim 1;
a container-closure system comprising a glass vial sealed with a low-permeability elastomeric stopper to prevent oxygen ingress and maintain sterility; and
instructions for use.

8. (canceled)

9. The pharmaceutical formulation according to claim 1, wherein the formulation is provided in vial pack sizes of 200 mg/5.26 mL, 1 g/26.3 mL, and 2 g/52.6 mL.

10. The pharmaceutical formulation according to claim 1, wherein the formulation is diluted with 0.9% NaCl.

11. The pharmaceutical formulation according to claim 10, wherein the gemcitabine is present in an amount of about 0.1 mg/mL in the diluted formulation.

12. A method of preparing a pharmaceutical formulation according to claim 1, comprising the steps of:
(a) dissolving an effective amount of gemcitabine hydrochloride in water for injection;
(b) adjusting the pH with sodium hydroxide to 2.0 to 3.0, and making up the volume using water for injection;
(c) filtering the solution with a sterile grade A 0.22 μm filter under aseptic conditions;
(d) filling the sterile filtered solution into a vial at the target fill volume range; and
(e) stoppering and sealing the vial with a flip-off seal;
wherein nitrogen purging is done during one or more of steps (a) to (e).

13. A method for treating:
(a) advanced ovarian cancer that has relapsed at least 6 months after completion of platinum-based therapy in combination with carboplatin;
(b) metastatic breast cancer in combination with paclitaxel;
(c) non-small cell lung cancer in combination with cisplatin; and/or
(d) pancreatic cancer;
comprising: administering an effective amount of a formulation according to claim 1 to a subject in need thereof;
wherein the formulation is provided in multi-dose vials in pack sizes of 200 mg/5.26 mL, 1 g/26.3 mL, and 2 g/52.6 mL.

14. A method for administering a stable multi-dose pharmaceutical injectable liquid formulation, comprising:
a) providing a formulation comprising: gemcitabine, or a pharmaceutically acceptable salt thereof, in a concentration of 38 mg/mL; an alkalizing agent; and a pharmaceutically acceptable fluid; wherein the formulation is stored in a multi-dose vial;
b) withdrawing a required dose under aseptic conditions from the multi-dose vial; and
c) administering the required dose to the patient;
d) storing the remaining formulation in the vial within the original carton at from about 2 °C to about 8 °C, for up to 14 days;
e) utilizing the remaining formulation according to the required dose within 14 days of first withdrawal to said patient and/or to another patient.

15. The method according to claim 14, wherein the formulation remains stable without the use of preservatives.

16. The method according to claim 14, wherein the multi-dose vial includes an elastomeric stopper to prevent contamination over multiple withdrawals.

17. The method according to claim 14, wherein the formulation is diluted with 0.9% Sodium Chloride Injection, USP before administration to achieve a final concentration of at least 0.1 mg/mL.

18. The method according to claim 17, wherein the formulation is used in oncological treatments for pancreatic, lung, or breast cancer patients.

19. A method for reducing drug wastage and ensuring safe administration of a multi-dose pharmaceutical injectable liquid formulation of gemcitabine, comprising:
a) providing a multi-dose vial containing a stable injectable liquid formulation comprising:
i) gemcitabine, or a pharmaceutically acceptable salt thereof, at a concentration of 38 mg/mL;
ii) an alkalizing agent; and
iii) a pharmaceutically acceptable fluid;
b) withdrawing a required dose under aseptic conditions and administering it to a patient;
c) storing the partially used multi-dose vial in the original carton at from about 2 °C to about 8 °C, for up to 14 days;
d) utilizing the remaining formulation according to the required dose within 14 days of first withdrawal;
wherein the 2′-Deoxy-2′,2′-difluorouridine concentration in the formulation does not exceed 1.5 wt. %;
wherein the total of other impurities in the formulation does not exceed 1.0 wt. %, as determined by HPLC analysis;
wherein the formulation remains sterile after multiple withdrawals; and
wherein the formulation does not have detectable degradation.

20. The method according to claim 19, wherein the remaining formulation is used for the same or different patient within 14 days of first use.

21. The method according to claim 19, wherein a printed label on the vial and carton, packaging label insert advises medical care workers about the multi-dose utilization, including injection of the unused portion within 14 days of first use, when stored at a temperature of from about 2 °C to about 8 °C.

22. The method according to claim 19, wherein drug wastage is avoided.
